(19) United States
(12) Patent Application Publication
(10) Pub. No.: US 2023/0346708 A1
(43) Pub. Date: Nov. 2, 2023

(54) STABLE FORMULATIONS OF ANTI-CANCER PEPTIDES

(71) Applicant: Oncolyze, Inc., New York, NY (US)

(72) Inventor: Steven EVANS, New York, NY (US)

(21) Appl. No.: 18/016,855

(22) PCT Filed: Dec. 28, 2020
(86) PCT No.: PCT/US2020/067161

§ 371 Date: Jan. 18, 2023

Related U.S. Application Data
(60) Provisional application No. 62/957,531, filed on Jan. 6, 2020.

(51) Int. Cl. A61K 9/19; A61K 47/12; A61K 47/26; C07K 14/47; A61K 9/08

(52) U.S. Cl. CPC A61K 9/19; A61K 47/12; A61K 47/26; C07K 14/4746; A61K 9/08; C07K 2319/01

(57) ABSTRACT

Stable, liquid formulations of anti-cancer peptides where the formulation contains about 1-75 mg/ml of anti-cancer peptides, about 5-30 mM acetate buffer, about 10-80 mg/ml mannitol, and about 0-30% sucrose, where the formulation has a pH of about 4.0-6.0, and wherein the anti-cancer peptides include an HDM-2 binding component and a membrane resident component.

The present invention is directed to pharmaceutical formulations comprising anti-cancer peptides, and to methods for making and using such formulations. The invention includes pharmaceutical formulations having increased stability.

### BACKGROUND OF INVENTION

Pharmaceutical formulations that contain proteins and/or peptides are prone to degradation due to the denaturation and aggregation of proteins during production of such formulations. One of the major issues with preparing pharmaceutical formulations that contain proteins is the formation of soluble and insoluble particles. This formation of soluble and insoluble particles worsens over time when such formulations are stored. In addition, pharmaceutical formulations that contain proteins can be unstable and lose bioactivity over time.

Therefore, there exists a need for developing technology for formulating pharmaceuticals that contain proteins and/or peptides that avoid the formation of soluble and insoluble particles and have increased physical, chemical and biological stability over time.

### SUMMARY OF THE INVENTION

Provided are stable, liquid formulations of anti-cancer peptides where the formulation contains about 1-75 mg/ml of anti-cancer peptides, about 5-30 mM acetate buffer, about 10-80 mg/ml mannitol, and about 0-30% sucrose, where the formulation has a pH of about 4.0-6.0, and wherein the anti-cancer peptides include an HDM-2 binding component and a membrane resident component.

According to the invention, the formulation preferably contains about 15-75 mg/ml of the anti-cancer peptides, and more preferably about 25-75 mg/ml of the anti-cancer peptides. The formulation contains preferably about 10-20 mM acetate buffer, and preferably about 20-60 mg/ml mannitol. The preferred pH of the formulation is about 5.0 to 6.0.

In one embodiment, the anti-cancer peptide contains SEQ ID NO: 48. The peptide can further contain any one or more of the sequences listed in TABLE 3.

According to the invention, the acetate buffer can comprise sodium acetate trihydrate, and the formulation can further comprise at least one of sodium chloride and sucrose.

In another embodiment, the formulation comprises about 25 mg/ml of the anti-cancer peptide and about 10 mM sodium acetate trihydrate, wherein the liquid formulation has a pH of about 5.1.

The formulation can also contain one or more of starch, glucose, dextrose, lactose, sucrose, gelatin, emulsifier, silica gel, sodium stearate, glycerol monostearate, polysorbate, sodium chloride, glycerol, propylene, glycol, and ethanol.

A stable, liquid formulation of anti-cancer peptides is also provided in which the formulation comprises 15-75 mg/ml of anti-cancer peptides comprising an HDM-2 binding component and a membrane resident component, 10-20 mM acetate buffer, 20-60 mg/ml mannitol, about 20% sucrose, about 100 mM NaCl, and about 0.1% polysorbate 20; wherein the formulation has a pH of about 4.0-6.0.

In one embodiment, the formulation comprises no particles greater than about 2.0 nm, no particles greater than about 3.0 nm, or no particles greater than about 5.0 nm.

In another embodiment, the formulation does not form a gel upon incubation at about 40° C. for two weeks.

According to the invention, less than about 20% of the anti-cancer peptide in the formulation is degraded after 2 years of storage at about 2-8° C., is degraded after 3 years of storage at about 2-8° C., or is degraded after 5 years of storage at about 2-8° C. Preferably, less than about 10% of the anti-cancer peptide in the formulation is degraded after 2 years of storage at about 2-8° C., is degraded after 3 years of storage at about 2-8° C., or is degraded after 5 years of storage at about 2-8° C. More preferably, less than about 5% of the anti-cancer peptide in the formulation is degraded after 2 years of storage at about 2-8° C., is degraded after 3 years of storage at about 2-8° C., or is degraded after 5 years of storage at about 2-8° C.

Provided are stable, lyophilized formulations of anti-cancer peptides, an HDM-2 binding component, a membrane resident component, acetate, and mannitol, where the formulation has a pH of about 4.0-6.0 and where the formulation is reconstitutable with a liquid to become a particle-free solution containing about 20-30 mg/ml concentration of the anti-cancer peptide within about 2 minutes or less.

In the stable, lyophilized formulation according to the invention, the particle-free solution comprises no particles greater than about 2.0 nm, no particles greater than about 3.0 nm, or no particles greater than about 5.0 nm.

In one embodiment, the formulation is stable at about 2-8° C. for at least three months, or at least six months or at least one year. In the stable, lyophilized formulation of the invention, less than about 20% of the anti-cancer peptide is degraded after about 2 years of storage at about 2-8° C. Preferably, less than about 20% of the anti-cancer peptide is degraded after about 3 years of storage at about 2-8° C., or about 5 years of storage at about 2-8° C. Preferably, less than about 10% of the anti-cancer peptide in the formulation is degraded after 2 years of storage at about 2-8° C. Preferably, less than about 10% is degraded after 3 years of storage at about 2-8° C., or is degraded after 5 years of storage at about 2-8° C. More preferably, less than about 5% of the anti-cancer peptide in the formulation is degraded after 2 years of storage at about 2-8° C., is degraded after 3 years of storage at about 2-8° C., or is degraded after 5 years of storage at about 2-8° C.

### BRIEF DESCRIPTION OF THE FIGURES

FIG. 1: Effect of NaCl on the initial time points by DLS.

FIG. 2: Effect of PNC-27 concentration by DLS

FIG. 3: Effect of Sodium Acetate concentration by DLS

FIG. 4: Effect of Mannitol concentration by DLS

### DETAILED DESCRIPTION

The present invention relates to formulations containing anti-cancer peptides, wherein the anti-cancer peptides include an HDM-2 binding component and a membrane resident component.

According to the invention, the anti-cancer peptide is PNC-27 or PNC-28 (SEQ ID NOs: 48 or 49) which are disclosed and described in, for example, U.S. application Ser. No. 14/470,488 filed on Aug. 27, 2014 and U.S. Pat. No. 9,539,327 issued on Jan. 10, 2017, both of which are hereby incorporated be referenced in their entirety in the application.

In one embodiment, the HDM-2 targeting component is a peptide that is selective for HDM-2. The peptide can be synthesized by any method known in the art. Furthermore, the peptide may include a functional group at the N-terminus or C-terminus that allows for conjugation to a small molecule or peptide. In this embodiment, the polypeptide may include alkynylene, alkoxy, azide, N-Hydroxysuccinimide Esters, imidoester, carbdiimides, maleimide, haloacetyl, pyridyl disulfide, and diazirine.

Examples of functional groups and reactions suitable for use in conjugation described above include:

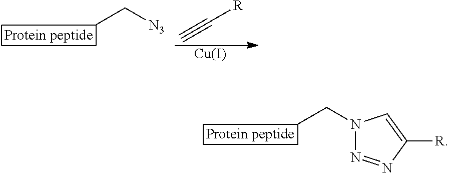

Such methods of conjugation are known in the art. For example, as described in Hermanson Bioconjugate Techniques, Third Edition (2013) (ISBN-10: 0123822394); the contents of which are incorporated herein by reference.

TABLE 1
HDM-2 Binding Peptides.

| SEQ ID NO: | HDM-2 TARGETING COMPONENT |
|---|---|
| 1 | PPLSQETFSDLWKLL |
| 2 | ETFSDLWKLL |
| 3 | MPRFMDYWEGLN |
| 4 | VQNFIDYWTQQF |
| 5 | TGPAFTHYWATF |
| 6 | IDRAPTFRDHWFALV |
| 7 | PRPALVFADYWETLY |
| 8 | PAFSRFWSDLSAGAH |
| 9 | PXFXDYWXXL |
| 10 | QPTFSDYWKLLP |
| 11 | PPL--TSFXEYWALLX-P |
| 12 | PPLSQTSFAEYWNLL |
| 13 | LTFEHYWAQLTS |
| 14 | TSFAEYWNLLSP |
| 15 | QETFSDLWKLLP |
| 16 | MPRFMDYWEGLN |
| 17 | QQMHLMSYAPGP |
| 18 | TIRPSTTMDSPT |
| 19 | YANPQMEKAFES |
| 20 | LTFEHYWAQLTS |
| 21 | LPNLTWALMPGA |
| 22 | YANPQMEKAFAS |
| 23 | LTFEHYWAQLTS |
| 24 | LLADTTHHRPWT |

In one embodiment, the HDM-2 targeting component is an antibody or antibody fragment. In one embodiment, the HDM-2 targeting component is an antibody that is selective for HDM-2. In one embodiment, the antibody fragment is an antibody fragment that is selective for HDM-2 For example, the antibody fragment includes scFv, sdAb, di-scFv. sdAb is a single domain antibody. scFv includes the VH and VL domains of an antibody and is connected by a linker. di-scFv includes two scFv molecules connect by a linker.

The antibody may be a monoclonal antibody or polyclonal antibody. In one embodiment, the antibody is selective for the surface exposed portions of HDM-2. In one embodiment, the antibody is selective for the p53 binding site of HDM-2. In one embodiment, the antibody is selective for residues 1-109 of HDM-2; 1-50 of HDM-2; 25-75 of HDM-2; or 50-109 of HDM-2.

In another embodiment, the antibody is a Camelid single domain antibody, or portions thereof. In one embodiment, Camelid single-domain antibodies include heavy-chain antibodies found in camelids, or VHH antibody. A VHH antibody of camelid (for example camel, dromedary, llama, and alpaca) refers to a variable fragment of a camelid single-chain antibody (See Nguyen et al, 2001; Muyldermans, 2001), and also includes an isolated VHH antibody of camelid, a recombinant VHH antibody of camelid, or a synthetic VHH antibody of camelid.

As used herein, antibody includes antibody fragments. In another embodiment, the HDM-2 targeting component is a small molecule. In one embodiment, the HDM-2 binding component is

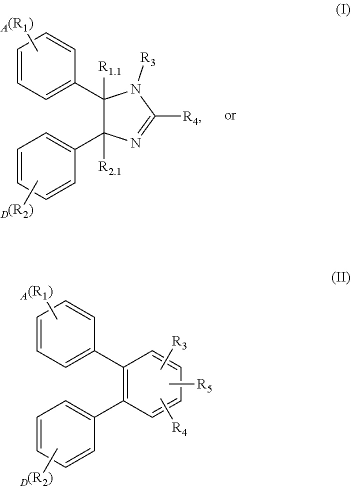

wherein R1, R1.1, R2, and R2.1 are independently H, halogen, lower alkylene, lower alkenylene, or lower alkynylene, optionally, with the proviso that when R1 or R2 is in the para position, R1 or R2 is not Br;

R3, R4, and R5 are independently H, halogen, lower alkylene, lower alkenylene, lower alkynylene, alkoxy, azide, N-Hydroxysuccinimide Esters, imidoester, carbdiimides, maleimide, haloacetyl, pyridyl disulfide, or diazirine,

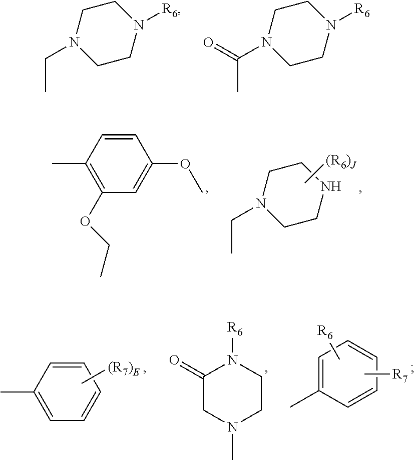

wherein R6 and R7 are independently H, halogen, lower alkylene, lower alkenylene, lower alkynylene, alkoxy, or

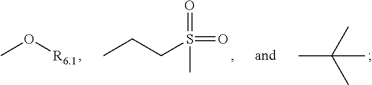

R6.1 is H, halogen, lower alkylene, lower alkenylene, lower alkynylene; A, D, E, G, and J are independently 1, 2, 3, 4, or 5.

In one embodiment, R₃, R₄, R₅, R₆, or R₇ includes a functional group which allows conjugation to the membrane resident component. Such functional groups are known in the art. For example, the functional groups include alkynylene, alkoxy, azide, N-Hydroxysuccinimide Esters, imidoester, carbdiimides, maleimide, haloacetyl, pyridyl disulfide, and diazirine. In one embodiment, R6 is C₂H₅O.

In one embodiment, R₃, R₄, R₅, R₆, or R₇ is linked or conjugated to a MRC described herein.

The above molecule may be synthesized by any method known in the art. See, for example. Vassilev et al., Science, 2004 Feb 6; 303(5659):844-8; and Zhao et al., BioDiscovery 2013.

In one embodiment, the above molecule may be conjugated to the N-terminus, C-terminus, lysine, cysteine, or tyrosine of membrane resident component polypeptide. In this embodiment, a membrane resident component polypeptide may include additional lysine, cysteine, or tyrosine residues at the N-terminus, C-terminus, or added to any of the polypeptides disclosed herein.

Examples of small molecule HDM-2 targeting components include

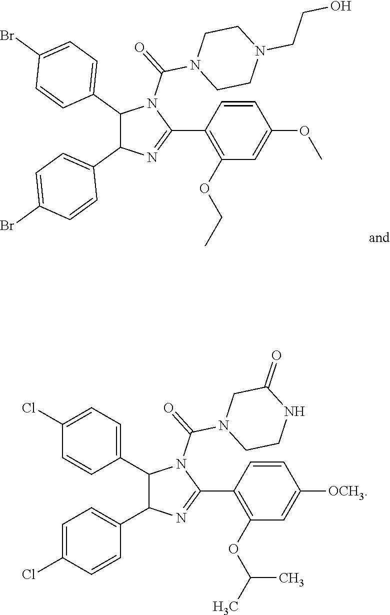 [SMILES: CCOc1cc(OC)ccc1C1=NC(c2ccc(Br)cc2)C(c2ccc(Br)cc2)N1C(=O)N1CCN(CCO)CC1 ; COc1ccc(C2=NC(c3ccc(Cl)cc3)C(c3ccc(Cl)cc3)N2C(=O)N2CCNC(=O)C2)c(OC(C)C)c1]

### Membrane Resident Component

In one embodiment, the membrane resident component (MRC) is a peptide or a membrane resident peptide (MRP). In one embodiment, the MRP may include a functional group that allows conjugation to a small molecule HDM-2 targeting component, as described above.

TABLE 2
Membrane Resident Peptides (MRP).

| SEQ ID | | |
|---|---|---|
| NO: | NAME | MRP |
| 25 | Membrane resident peptide | KKWKMRRNQFWVKVQRG |
| | (MRP), reverseomer of | |
| | Antennapedia | |
| 26 | peptide from cytochrome P450 | MPFSTGKRIMLGE |
| | (aka “X13”) | |
| 27 | HIV-1 TAT(47-60), membrane | YGRKKRRQRRRPPQ |
| | resident peptide | |
| 28 | D-TAT, membrane resident | GRKKRRQRRRPPQ |
| | peptide | |
| 29 | R-TAT G(R)₉PPQ, membrane | GAAAAAAAAAPPQ |
| | resident peptide | |
| 30 | SV40-NLS, membrane | PKKKRKV |
| | resident peptide | |
| 31 | nucleoplasmin-NLS, | KRPAAIKKAGQAKKKK |
| | membrane resident peptide | |
| 32 | HIV REV (34-50), membrane | TRQARRNRRRRWRERQR |
| | resident peptide | |
| 33 | FHV (35-49) coat, membrane | RRRRNRTRRNRRRVR |
| | resident peptide | |
| 34 | BMV GAG (7-25), membrane | KMTRAQRRAAARRNRWTAR |
| | resident peptide | |
| 35 | HTLV-II REX 4-16, | TRRQRTRRARRNR |
| | membrane resident peptide | |
| 36 | CCMV GAG (7-25), | KLTRAQRRAAARKNKRNTR |
| | membrane resident peptide | |
| 37 | P22 N (14-30), membrane | NAKTRRHERRRKLAIER |
| | resident peptide | |
| 38 | LAMBDA N(1-22), membrane | MDAQTRRRERRAEKQAQWKAAN |
| | resident peptide | |
| 39 | Phi N (12-29), membrane | TAKTRYKARRAELIAERR |
| | resident peptide | |
| 40 | YEAST PRP6 (129-124), | TRRNKRNRIQEQLNRK |
| | membrane resident peptide | |
| 41 | HUMAN U2AF, membrane | SQMTRQARRLYV |
| | resident peptide | |
| 42 | HUMAN C-FOS | KRRIRRERNKMAAAKSRNRRRELTDT |
| | (139-164), membrane resident | |
| | peptide | |
| 43 | HUMAN C-JUN | RIKAERKRMRNRIAASKSRKRKLERIA R |
| | (252-279), membrane resident | |
| | peptide | |
| 44 | YEAST GCN4, membrane | KRARNTEAARRSRARKLQRMKQ |
| | resident peptide | |
| 45 | Example membrane resident | KLALKLALKALKAALKLA |
| | peptide (MRP) | |
| 46 | p-vec, membrane resident | LLIILRRRIRKQAKAHSK |
| | peptide | |
| 47 | (Arg)₈ or any poly-R from | RRRRRRRR |
| | (R)₄-(R)₁₆, membrane resident | |
| | peptide | |

In one embodiment, the MRC is a small molecule. For example, the MRC is

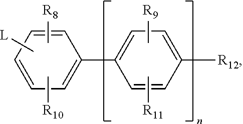

wherein L is linked or conjugated to a HDM-2 targeting component; n is 0, 1, 2, 3, 4, 5, 6, 7; in one embodiment, n is an even number between 0 and 100.

L may be a funcitonal group which allows conjugation to a similarly functionalized molecule or capable of reacting with L, when L is: (Z)ₘNR₁₅R₁₆ where Z is a hydrocarbyl group and m is 0 or 1; where R₁₅ and R₁₆ are each independently H, CO(CH₂)ⱼQ₁ or C═S(NH)(CH2)ₖQ₂ where j and k are each independently 0, 1, 2, 3, 4, or 5, and Q₁ and Q₂ are each independently selected from COOH, a chromophore

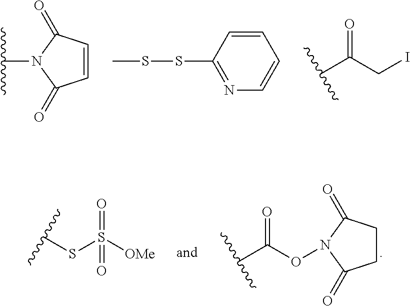

R₈, R₉, R₁₀, and R₁₁ are each independently

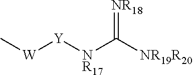

where Y is an alkylene, alkenylene, or alkynylene group, each of which may be optionally substituted with one or more substituents selected from alkyl, halo, CF₃, OH, alkoxy, NH₂, CN, NO₂, and COOH; W is absent or is O, S, or NH; R₁₇, R₁₈, R₁₉, and R₂₀ are each independently selected from H, alkyl, aryl, and a protecting group P1. Protecting groups are commonly known in the art. An example of a suitable protecting group includes tert-Butyloxycarbonyl (BOC).

In one embodiment, R₈, R₉, R₁₀, and R₁₁ are each

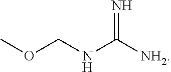

### R₁₂ is

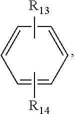

wherein
R₁₃ and R₁₄ are each independently

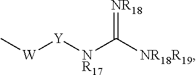

where Y is an alkylene, alkenylene, or alkynylene group, each of which may be optionally substituted with one or more substituents selected from alkyl, halo, CF₃, OH, alkoxy, NH₂, CN, NO₂, and COOH; W is absent or is O, S, or NH; R₁₇, R₁₈, R₁₉, and R₂₀ are each independently selected from H, alkyl, aryl, and a protecting group P1.

The above molecule may be synthesized by any method known in the art. See, for example, Okuyama et al., Nature Methods, January 2007.

In another embodiment, the membrane resident component includes a polypeptide configured to conjugate to the compound of formula I or formula II. In this embodiment, the polypeptide may include alkynylene, alkoxy, azide, N-Hydroxysuccinimide Esters, imidoester, carbdiimides, maleimide, haloacetyl, pyridyl disulfide, and diazirine.

### HDM-2 and MRC

The HDM-2 targeting component and MRC as described above are covalently linked. They may be linked directly or by way of a linker. Compositions having a HDM-2 targeting component that are conjugated or covalently linked to a MRC or MRP define the compositions disclosed herein.

The anti-cancer peptides of the present invention include a HDM-2 targeting component and a membrane resident component (MRP). In the case of the HDM-2 targeting component is a polypeptide, it may be conjugated to the N-terminus or the C-terminus of the MRP.

The anti-cancer peptides of the present invention may include, for example, PNC-27 and PNC-28. The HDM-2 targeting components may be, for example, the residues of p53 which bind to HDM-2. Both PNC-27 and PNC-28 are examples of p53-derived peptides from the human double minute binding domain (HDM-2) that are attached to MRP. These peptides induce tumor cell necrosis of cancer cells, but not normal cells. The anti-cancer activity and mechanism of PNC-28 (p53 aa17-26-MRP) was specifically studied by the inventor of the present invention as against human pancreatic cancer, though uses and applications are included with the various methods of the present invention.

The MRC is necessary for this action since expression of the naked p53 sequence without MRC in cancer cells causes wild-type p53-dependent apoptosis, or programmed cell death, not tumor cell necrosis.

In one embodiment, the MRC is an MRP. Preferably, the MRP includes predominantly positively charged amino acid residues since a positively charged leader sequence, which may stabilize the alpha helix of a subject peptide. Examples of MRPs which may be useful to the HDM-2 targeting components of the present invention are described in Futaki, S. et al (2001) Arginine-Rich Peptides, J. Biol. Chem. 276,:5836-5840, and include but are not limited to the

MRPs listed in TABLE 2. The MRP may be, for example, peptides included in SEQ ID NO: 25-47. The numbering of the amino acid residues making up the MRP is indicated before the name of the component in most of the examples in most of the sequence listings, and in Table 2.

In one embodiment, the polypeptide HDM-2 targeting component and the MRP may be independently stabilized.

TABLE 3
Anti Cancer Peptides

| SEQ | | |
|---|---|---|
| ID: | Name | Sequence |
| 48 | PNC-27 | PPLSQETFSDLWKLLKKWKMRRNQFWVKVQRG |
| 19 | PNC-28 | ETFSDLWKLLKKWKMRRNQFWVKVQRG |
| 50 | PNC-29 | MPFSTGKRIMLGEKKWKMRRNQFWVKVQRG |
| 51 | PNC-7 | TIEDSYRKQVVIDKKWKMRRNQFWVKVQRG |
| 52 | ras-p21 residues | TIEDSYRKQVVID |
| | 35-47 | |
| 53 | Kozak sequence | GCCACCATGG |
| 54 | sense strand | AGTCGAATTCGCCACCATGGAAACATT |
| | sequence of cDNA | TTCAGACCTATGGAAACTACTTTGAGCGGCCGCAGTC |
| | encoding the p53 | |
| | 17-26 sequence | |
| 55 | residues 17-26 of | ETFSDLWKLL |
| | HDM-2 binding | |
| | domain of p53 | |
| 56 | PNC-21 | PPLSQETFS |

In one embodiment, the HDM-2 targeting component and the MRC are small molecules. For example, the following structure is an example of a small molecule HDM-2 targeting component bound to an MRC.

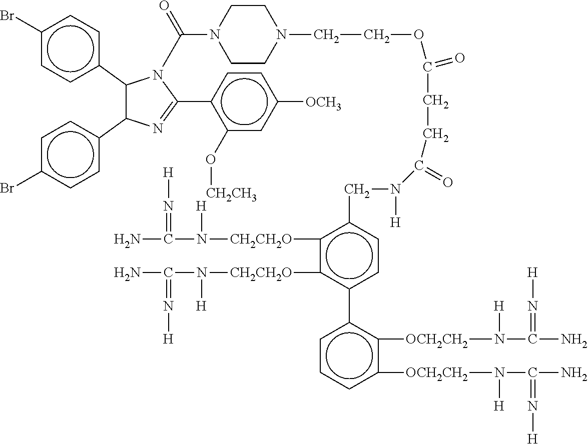

In another embodiment, the HDM-2 targeting component is an antibody, as described above, and the MRC is a peptide (MRP). The C-terminus or the N-terminus of the MRP may be conjugated to the HDM-2 targeting antibody.

In another embodiment, the HDM-2 targeting component is an antibody, as described above, and the MRC is a small molecule.

In another embodiment, the HDM-2 targeting component is a peptide and the MRC is a small molecule.

In another embodiment, the HDM-2 targeting component is a small molecule and the MRC is a peptide (MRP).

The HDM-2 targeting component and the MRC may be attached by way of a linker. The linker may be a peptide linker, macromolecular linker, chemical linker, or polymeric linker.

Peptide linker may have a maximum length of 6, 7, 8, 9, 10, 11, 12, 13, 14, 15, 20, 25, 50, or 100 amino acid residues. The peptide linker may have a minimum of 1, 2, 3, 4, 5, 6, 7, 8, 9, 10, 25, or 50 amino acid residues.

In one embodiment, the linker is polyglutamic acid (PGA).

Examples of other suitable linkers include polyethylene glycol (PEG). The PEG may be branched or linear and each PEG may have a molecular weight between about 200 and 100,000

Daltons. In one embodiment, the PEG has a minimum molecular weight of 400, 500, 1,000, 2,500, 5,000, or 10,000. In one embodiment, the PEG has a maximum molecular weight of 1,000, 2,500, 5,000, 10,000, 25,000, 50,000, 75,000, or 100,000.

In one embodiment, the PEG includes multi-arm PEG.

In one embodiment, the linker is polysarcosine (PSR) polyoxazolines, polyactides, poly lactide-co-glycolide (PLGA), or chitosan.

The linker may be the result of a conjugation reaction between the functional group on the HDM-2 targeting component and the MRC.

The synthetic peptides of the present invention may be synthesized by a number of known techniques. For example, the peptides may be prepared using the solid-phase technique initially described by Merrifield (1963) in J. Am. Chem. Soc. 85:2149-2154. Other peptide synthesis techniques may be found in M. Bodanszky et al. Peptide Synthesis, John Wiley and Sons, 2d Ed., (1976) and other references readily available to those skilled in the art. A summary of polypeptide synthesis techniques may be found in J. Stuart and J. S. Young, Solid Phase Peptide Synthesis, Pierce Chemical Company, Rockford, Ill., (1984). Peptides may also be synthesized by solid phase or solution methods as described in The Proteins, Vol. II, 3d Ed., Neurath, H. et al., Eds., pp. 105-237, Academic Press, New York, N.Y. (1976). Appropriate protective groups for use in different peptide syntheses are described in the texts listed above as well as in J. F. W. McOmie, Protective Groups in Organic Chemistry, Plenum Press, New York, N.Y. (1973). The peptides of the present invention may also be prepared by chemical or enzymatic cleavage from larger portions of the p53 protein or from the full length p53 protein. Likewise, membrane-resident sequences for use in the synthetic peptides of the present invention may be prepared by chemical or enzymatic cleavage from larger portions or the full length proteins from which such leader sequences are derived.

The synthetic small molecules of the present invention may be synthesized by a number of known techniques. For example, a molecule according to the present invention may be synthesized as follows.

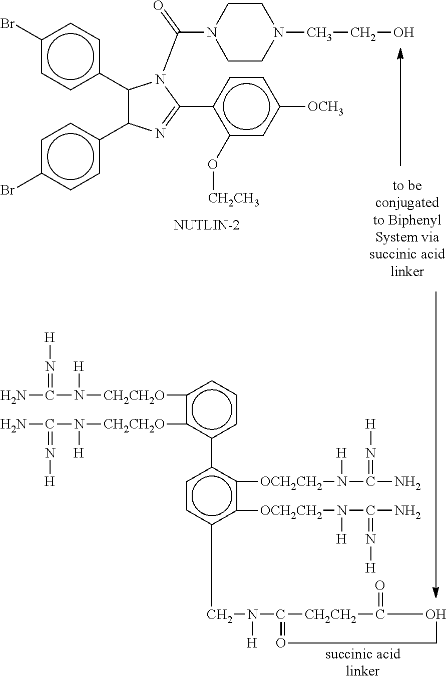

Additionally, the peptides of the present invention may also be prepared by recombinant DNA techniques. For most amino acids used to build proteins, more than one coding nucleotide triplet (codon) can code for a particular amino acid residue. This property of the genetic code is known as redundancy. Therefore, a number of different nucleotide sequences may code for a particular subject peptide selectively lethal to malignant and transformed mammalian cells. The present invention also contemplates a deoxyribonucleic acid (DNA) molecule that defines a gene coding for, i.e., capable of expressing a subject peptide or a chimeric peptide from which a peptide of the present invention may be enzymatically or chemically cleaved.

Examples of anti-cancer peptides include PNC-27 and PNC-28 and variations thereof including amino acid substitutions including with D-amino acids, and with the attachment of other peptide-stabilizing structures such as leupeptin and polyarginine, and SLH-1 and SLH-1 and variations thereof including amino acid substitutions including with D-amino acids, and with the attachment of other peptide-stabilizing structures such as leupeptin and polyarginine. Such peptides are found, for example, in U.S. Pat. No. 9,765,117 which issued on Sep. 19, 2017 and whereby said disclosure in its entirety is hereby incorporated by reference in this application.

In one aspect, the present invention is directed to a stable liquid formulation of an anti-cancer peptide, said formulation comprising: 15-75 mg/ml of an anti-cancer peptide comprising an HDM-2 binding component and a membrane resident component, 10-20 mM acetate buffer, 20-60 mg/ml mannitol, and 0-30% sucrose; at a pH of about 4.0-6.0.

In one aspect, the present invention is directed to a stable lyophilized formulation of an anti-cancer peptide and methods of preparing the same.

Safe handling and administration of formulations comprising anti-cancer peptidess represent significant challenges to pharmaceutical formulators. Anti-cancer peptides possess unique chemical and physical properties that present stability problems: a variety of degradation pathways exist for anti-cancer peptides, implicating both chemical and physical instability. Chemical instability includes deamination, aggregation, clipping of the peptide backbone, and oxidation of methionine residues. Physical instability encompasses many phenomena, including, for example, aggregation.

Chemical and physical stability can be promoted by removing water from the anti-cancer peptides. Lyophilization (freeze-drying under controlled conditions) is commonly used for long-term storage of anti-cancer peptides. The lyophilized anti-cancer peptides are substantially resistant to degradation, aggregation, oxidation, and other degenerative processes while in the freeze-dried state. The lyophilized anti-cancer peptides are normally reconstituted with water optionally containing a bacteriostatic preservative (e.g., benzyl alcohol) prior to administration.

### Definitions

The term “carrier” includes a diluent, adjuvant, excipient, or vehicle with which a composition is administered. Carriers can include sterile liquids, such as, for example, water and oils, including oils of petroleum, animal, vegetable or synthetic origin, such as, for example, peanut oil, soybean oil, mineral oil, sesame oil and the like.

The term “excipient” includes a non-therapeutic agent added to a pharmaceutical composition to provide a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The term “lyophilized” or “freeze-dried” includes a state of a substance that has been subjected to a drying procedure such as lyophilization, where at least 50% of moisture has been removed.

The phrase “bulking agent” includes a compound that is pharmaceutically acceptable and that adds bulk to a lyo cake (the porous and spongy structure like material resulting from lyophilization process). Generally, acceptable bulking agents known to the art include, for example, carbohydrates, including simple sugars such as dextrose, ribose, fructose and the like, sugar alcohols such as mannitol, inositol and sorbitol, disaccharides including trehalose, sucrose and lactose, naturally occurring polymers such as starch, dextrans, chitosan, hyaluronate, anti-cancer peptidess (e.g., gelatin and serum albumin), glycogen, and synthetic monomers and polymers. In the formulations of the invention, PEG 3350 is an organic co-solvent which is used to stabilize the anti-cancer peptides when agitated, mixed, or handled, and as a bulking agent to help produce an acceptable bulk.

The term “lyoprotectant” includes a substance that may be added to a freeze-dried or lyophilized formulation to help maintain anti-cancer peptides structure when freeze-dried or lyophilized.

A “preservative” includes a bacteriostatic, bacteriocidal, fungistatic or fungicidal compound that is generally added to formulations to retard or eliminate growth of bacteria or other contaminating microorganisms in the formulations. Preservatives include, for example, benzyl alcohol, phenol, benzalkonium chloride, m-cresol, thimerosol, chlorobutanol, methylparaben, propylparaben and the like. Other examples of pharmaceutically acceptable preservatives can be found in the USP.

### Lyophilization and Lyophilized Formulations

In one aspect of the invention, a pharmaceutically acceptable formulation comprising anti-cancer peptide(s)_{[se1]} is provided, wherein the formulation is a freeze-dried or lyophilized formulation. Lyophilized formulations can be reconstituted into solutions, suspensions, emulsions, or any other suitable form for administration or use. Lyophilized formulations are typically first prepared as liquids, then frozen and lyophilized. The total liquid volume before lyophilization can be less, equal to, or more than, the final reconstituted volume of the lyophilized formulation. The lyophilization process is well known to those of ordinary skill in the art, and typically includes sublimation of water from a frozen formulation under controlled conditions.

Lyophilized formulations can be stored at a wide range of temperatures. Lyophilized formulations may be stored below 25° C., for example, refrigerated at 4° C., or at room temperature (e.g., approximately 25° C.). Preferably, lyophilized formulations are stored below about 25° C., more preferably, at about 4-20° C.; below about 4° C.; below about −20° C.; about −40° C.; about −70° C., or about −80° C.

Lyophilized formulations are typically reconstituted prior to use by addition of an aqueous solution to dissolve the lyophilized formulation. A wide variety of aqueous solutions can be used to reconstitute a lyophilized formulation. Preferably, lyophilized formulations are reconstituted using water. Lyophilized formulations are preferably reconstituted with a solution consisting essentially of water (e.g., USP WFI, or water for injection) or bacteriostatic water (e.g., USP WFI with 0.9% benzyl alcohol). However, solutions comprising buffers and/or excipients and/or one or more pharmaceutically acceptable carries can also be used.

Freeze-dried or lyophilized formulations are typically prepared from liquids, such as, for example, solutions, suspensions, emulsions, and the like. Thus, the liquid that is to undergo freeze-drying or lyophilization preferably comprises all components desired in a final reconstituted liquid formulation. As a result, when reconstituted, the freeze-dried or lyophilized formulation will render a desired liquid formulation upon reconstitution. A preferred liquid formulation used to generate a freeze-dried or lyophilized formulation comprises a anti-cancer peptides in a pharmaceutically effective amount, a buffer, a stabilizer, and a bulking agent. Freeze-dried or lyophilized formulations preferably comprise histidine, since histidine, in comparison to phosphate, is more effective at stabilizing the anti-cancer peptides when the anti- cancer peptides is lyophilized. Organic co-solvents, such as PEG 3350, are used to stabilize the anti-cancer peptides when agitated, mixed, or handled. A lyoprotectant is preferably used in freeze-dried or lyophilized formulations. Lyoprotectants help to maintain the secondary structure of anti-cancer peptidess and/or peptides when freeze-dried or lyophilized. Preferred example lyoprotectants are mannitol, glycine and sucrose, which may be used together.

### Stable Liquid Formulations

In one aspect, the invention provides a stable pharmaceutically acceptable formulation comprising anti-cancer peptides, wherein the formulation is a liquid formulation. Preferably, the liquid formulation comprises a pharmaceutically effective amount of the anti-cancer peptides. The formulation can also comprise one or more pharmaceutically acceptable carriers, buffers, bulking agents, stabilizers, preservatives, and/or excipients. An example of a pharmaceutically acceptable liquid formulation comprises anti-cancer peptides in a pharmaceutically effective amount, a buffer, a co-solvent, and one or more stabilizers.

A preferred liquid formulation comprises phosphate buffer, an organic co-solvent, and one or more thermal stabilizers to minimize formation of aggregates and low molecular weight products when stored, and about 10 mg/ml to about 50 mg/ml of anti-cancer peptides, wherein the formulation is at a pH of about 6.0; optionally polysorbate may be present (e.g., 0.1% polysorbate 20). Although either NaCl or sucrose can be used as a stabilizer, a combination of NaCl and sucrose has been established to stabilize the anti-cancer peptides more effectively than either individual stabilizer alone.

Stability is determined in a number of ways at specified time points, including determination of pH, visual inspection of color and appearance, determination of total anti-cancer peptides content by methods known in the art, e.g., UV spectroscopy, SDS-PAGE, size- exclusion HPLC, bioassay determination of activity, isoelectric focusing, and isoaspartate quantification.

Formulations, whether liquid or freeze-dried and lyophilized, can be stored in an oxygen-deprived environment. Oxygen-deprived environments can be generated by storing the formulations under an inert gas such as, for example, argon, nitrogen, or helium.

### Preparation of the Formulation

After the anti-cancer peptides of interest are prepared as described above, the pharmaceutical formulation comprising the anti-cancer peptides is prepared. The formulation development approach is as follows: selecting the optimum solution pH, selecting buffer type and concentration, evaluating the effect of various excipients of the liquid and lyophilized stability, and optimizing the concentration of the screened excipients using an I-optimal experimental design (Statistics for Experimental, Box, George E. P. John Wiley and Sons, Inc., 1978).

The following criteria are important in developing stable lyophilized anti-cancer peptide products. Anti-cancer peptides unfolding during lyophilization should be minimized. Various degradation pathways should be minimized. Glass transition temperature (Tg) should be greater than the product storage temperature. Residual moisture should be low (<1% by mass). A strong and elegant cake structure should be obtained. A preferred shelf life should be at least 3 months, preferably 6 months, more preferably 1 year at room temperature (22 to 28° C.). A reconstitution time should be short, for example, less than 5 minutes, preferably less than 2 minutes, and more preferably less than 1 minute. When the lyophilized product is reconstituted, the reconstituted sample should be stable for at least 48 hours at 2-8° C.

The formulations of this invention minimize the formation of anti-cancer peptide aggregates and particulates in reagents and insure that the anti-cancer peptides in solution maintains its bioactivity over time. The formulations comprise a sterile, pharmaceutically acceptable lyophilized formulation prepared from an aqueous pre-lyophilized formulation comprising anti-cancer peptides in a buffer having a neutral or acidic pH (pH 5.5-6.5), a surfactant, and a polyol. The preferred formulation additionally contains a bulking agent, and/or a tonicity modifier.

A buffer of pH 5.5-6.5 is used in the formulation. Examples of buffers that control the pH in this range include acetates, succinate (such as sodium succinate), gluconate, histidine, citrate and other organic acid buffers. Acetate is a preferred buffer for subcutaneous, intramuscular and peritoneal injection. In particular, sodium acetate tri-hydrate is preferred. Sodium succinate buffer is less preferred because it does not have a good buffer capacity at low strength. To increase the buffer strength of sodium succinate, the amount of the excipients will have to be decreased in order to maintain the osmolarity in a desired range. If the lyophile is to be reconstituted with half of the fill volume, then the desired osmolarity of the pre-lyophilized (fill) liquid is about 140-160 mOsm. Citrate buffer is also less preferred because it causes a painful reaction when injected subcutaneously.

A surfactant is added to the anti-cancer peptides formulation. Exemplary surfactants include nonionic surfactants such as polysorbates (e.g. polysorbates 20, 80, such as Tween ®20, Tween®80) or poloxamers (e.g. poloxamer 188). The amount of surfactant added is such that it reduces aggregation of the formulated anti-cancer peptides and/or minimizes the formation of particulates in the formulation and/or reduces anti-cancer peptides adsorption onto the container. The surfactant also reduces the reconstitution time of the lyophilized formulation. For example, the surfactant is present in the formulation in an amount from about 0.001% to about 0.5%, preferably from about 0.005% to about 0.1% and most preferably from about 0.01% to about 0.05%.

A polyol, which acts as a tonicifying agent and a cryoprotector/lyoprotector, is included in the formulation. Mannitol is a preferred polyol for this invention. In another embodiment, the polyol is a non-reducing sugar, such as sucrose or trehalose. In the present invention, the polyol such as mannitol, is the primary stabilizer against anti-cancer peptides aggregation, and it also plays an important role in reducing the reconstitution time of the lyophilized formulation to a particle-free solution. The polyol is added to the formulation in an amount that may vary with respect to the desired tonicity of the formulation. Preferably, the lyophilized formulation after reconstitution is isotonic; however, hypertonic or hypotonic formulations may also be suitable. Suitable concentrations of the polyol, such as mannitol, in the pre-lyophilized formulation are in the range from about 10-50 mg, preferably in the range from about 20-40 mg.

A bulking agent that provides good lyophilized cake properties, such as serine, glycine, mannitol, can be optionally added to the present composition. These agents also contribute to the tonicity of the formulations and may provide protection to the freeze-thaw process and improve long-term stability. A preferred bulking agent is serine at a concentration about 15-55 mM, and preferably about 20-30 mM. Another preferred bulking agent is mannitol, at a concentration about 10-55 mM, and preferably about 20-45 mM. The addition of serine or mannitol to the pre-lyophilized formulation reduces the concentration of polyol required for stabilizing the anti-cancer peptides, for example, to 30-180 mM and preferably 80-130 mM.

Tonicity modifiers such as salts (e.g., NaCl, KCl, MgCl2, CaCl2) can be added to the formulation to control osmotic pressure.

Exemplary pre-lyophilized compositions are formulations comprising a peptide at about 50 mg/ml or greater, about 10-20 mM acetate (pH 5.5-6.5), about 0.005-0.03% polysorbate 20 or 80, and one of the following combinations of excipients: (a) 100-200 mM sucrose, (b) 110-130 mM sucrose and 20-45 mM mannitol, (c) 100-130 mM sucrose and 15-55 mM serine, and (d) 7-55 mM serine, 80-130 mM sucrose, and 10-55 mM mannitol. The above pre-lyophilized formulation is lyophilized to form a dry, stable powder, which can be easily reconstituted to a particle-free solution suitable for administering to humans.

Lyophilization is a freeze drying process that is often used in the preparation of pharmaceutical products to preserve their biological activity. The liquid composition is prepared, then lyophilized to form a dry cake-like product. The process generally involves drying a previously frozen sample in a vacuum to remove the ice, leaving the non-water components intact, in the form of a powdery or cake-like substance. The lyophilized product can be stored for prolonged periods of time, and at elevated temperatures, without loss of biological activity, and can be readily reconstituted into a particle-free solution by the addition of an appropriate diluent. An appropriate diluent can be any liquid which is biologically acceptable and in which the lyophilized powder is completely soluble. Water, particularly sterile, pyrogen-free water, is a preferred diluent, since it does not include salts or other compounds which may affect the stability of the anti-cancer peptides. The advantage of lyophilization is that the water content is reduced to a level that greatly reduce the various molecular events which lead to instability of the product upon long-term storage. The lyophilized product is also more readily able to withstand the physical stresses of shipping. The reconstituted product is particle free, thus it can be administered without prior filtration.

The liquid formulation can be lyophilized using appropriate drying parameters. The following drying parameters are preferred: a primary drying phase temperature of about −20° C. to −50° C. and pressure between about 80 mTorr to about 120 mTorr; and a secondary drying phase at ambient temperature, and pressure between about 80 mTorr to 120 mTorr.

This lyophilized product retains the stability of activity of the anti-cancer peptides, and prevents the anti-cancer peptides intended for administration to human subjects from physical and chemical degradation in the final product.

The lyophilized product is rehydrated at the time of use in a diluent (e.g., sterile water or saline) to yield a particle-free solution. The reconstituted anti-cancer peptides solution is particle-free even after prolonged storage of the lyophilized cake at ambient temperature. The reconstituted solution is administered parenterally, preferably intravenously or subcutaneously, to the subject.

An important characteristic of the lyophilized product is the reconstitution time or the time taken to rehydrate the product. To enable very fast and complete rehydration, it is important to have a cake with a highly porous structure. The cake structure is a function of a number of parameters including the anti-cancer peptides concentration, excipient type and concentration, and the process parameters of the lyophilization cycle. Generally, the reconstitution time increases as the anti-cancer peptides concentration increases, and thus, a short reconstitution time is an important goal in the development of high concentration lyophilized anti-cancer peptides formulations. A long reconstitution time can deteriorate the product quality due to the longer exposure of the anti-cancer peptides to a more concentrated solution. In addition, at the user end, the product cannot be administered until the product is completely rehydrated. This is to ensure that the product is particulate-free, the correct dosage is administered, and its sterility is unaffected. Thus, quick rehydration offers more convenience to the patients and the physicians.

In lyophilized products, the desired dosage can be obtained by lyophilizing the formulation at the target anti-cancer peptides concentration and reconstituting the product with the same volume as that of the starting fill volume. The desired dosage can also be obtained by lyophilizing a larger volume of a diluted formulation, and reconstituting it with a less volume. For example, if a desired product dosage is 100 mg of anti-cancer peptides in 1 mL of the formulation, the formulations can be lyophilized with the following liquid configurations: 1 mL of 100 mg/mL, 2 mL of 50 mg/ml, or 4 mL of 25 mg/mL anti-cancer peptides formulation. In all cases, the final product can be reconstituted with 1 mL diluent to obtain the target anti-cancer peptides concentration of 100 mg/mL. However, as the anti-cancer peptides concentration in the pre-lyophilized formulation is reduced, the fill volume increases proportionately. This correspondingly increases the length of the lyophilization cycle (especially the primary drying time), and thus significantly adds to the cost of the product. For example, if 1 mL fill volume (1 mm height in vial) of frozen material takes approximately 1 hour to sublimate its free water, then 10 mL fill volume (10 mm height) of frozen product will take approximately 10 hours of primary drying time. Therefore, it is advantageous to have a concentrated pre-lyophilized formulation (with anti-cancer peptides greater than 50 mg/mL) such that the lyophilization process will be more efficient.

Embodiments of this invention are described herein, including the best mode known to the inventor for carrying out the invention. Variations of those embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The present invention is described by reference to the following examples, which are offered by way of illustration and are not intended to limit the invention in any manner. Standard techniques well known in the art or the techniques specifically described below are utilized.

### EXAMPLES

### Components and Composition

The proposed quantitative composition, component function, and quality standards of PNC-27 drug product are shown in Table 1 . PNC-27 drug product is a clear, colorless, sterile, isotonic pH 5.0 solution containing 25 mg/mL PNC-27 drug substance, acetate as buffer, and mannitol to control tonicity. The primary packaging for PCN-27 drug product is a 2 mL USP Type I clear glass vials with a Flurotec-coated butyl rubber stopper and aluminum crimp cap. Each vial will contain 2 mL of PNC-27 drug product solution. The label claim of 25 mg/mL is based on the free base PNC-27 peptide content. The route of administration will be infusion using either 5% dextrose (D5W) or normal 0.9% saline (D5NS). PNC-27 drug product will be stored frozen (−20° C.) and thawed prior to use, or stored at 5° C.

TABLE 4
Composition of PNC-27 Drug Product (25 mg/mL)

| Component | Function | Specification | Quantity | |
|---|---|---|---|---|
| PNC-27^{a} | Active ingredient | GMP (in house) | 25 | mg |
| Sodium Acetate Tri- | Buffering agent | USP | 1.36 | mg |
| Hydrate (10 mM) | | | | |
| Mannitol | Tonicity adjustment | USP | 40 | mg |
| 1N NaOH or HCl | pH 5.0 adjustment | USP | To adjust | |
| Water for injection | Vehicle | USP | Qs 1 mL^{b} | |
| Totals mL | | | 1 | mL |

^{a}PNC-27 quantities shown are weighed out on a free base content using the calculation 100%-acetate-water-residual solvent-related substances. For GJ1006 lot drug substance 1.15 mg of PNC-27 acetate salt corresponds to 1.00 mg PNC-27 as free base.
^{b}Formulations have been prepared on a weight basis but PNC-27 peptide content is determined by HPLC assay against an external reference standard with an assigned purity and reported as mg/ml.

### pH Stability

The effect of pH on PNC-27 chemical and physical stability was evaluated to determine the optimized pH for PNC-27 solution formulation. A set of 5 formulations at 25 mg/mL in water were prepared at pH 5.1, 5.7, 7.2, 8.0, and 8.8. The formulations were stored at between 2-8° C. and 40° C. and analyzed for appearance (solution vs. gel) and assay content at 1 and 2 week time points. Formulation details and tabulated data are provided in Table 2.

### Chemical Stability (Assay)

PNC-27 (25 mg/mL) in water is chemically stable at 2-8° C. by HPLC w/w assay up to 2 weeks at pHs 5.0-8.8. From a content perspective the solutions at pH 5.1 and 5.7 at 2-8° C. exhibited better stability than those at higher pH. The pH 8.8 sample showed a 1% loss in assay vs. pH of 5.1. Samples stored at 40° C. below pH 7.2 were stable vs. PNC-27 content as well as physical appearance. Samples stored at 40° C. above pH 7.2 gelled after 1 week and were not evaluated for PNC-27 content.

### Physical Stability (Appearance)

PNC-27 (25 mg/mL) in water is physically stable and remains a free-flowing solution at pH 5.1 and pH 5.7 at both 2-8° C. and 40° C. over 2 weeks. At pH 7.0, solutions at 2-8° C. are stable at 2 weeks but gelled at 40° C. after 1 week. At pH 7.2, solutions at 2-8° C. are stable at 2 weeks but gelled and precipitated at 40° C. after 1 week. At pH 8.6, solutions at 2-8° C. are also stable at 2 weeks but gelled and precipitated at 40° C. after 1 week.

### Conclusions

The data indicate that 25 mg/mL PNC-27 is chemically stable at a wide range of pHs for at least 2 weeks at 40° C. At 25 mg/mL physical stability (gelling) was found to occur at pH>5.7 after 2 weeks at 40° C. Based on this data a pH 5 was chosen for further development. Dynamic Light Scattering (DLS) is a more sensitive way to measure physical instability of aggregation in advance of visual appearance of gelling and is a critical test method to be employed in predicting the physical stability of PNC-27 drug solutions. Some of this data is presented in the following sections.

### Selection of Buffer and Tonicity Agent

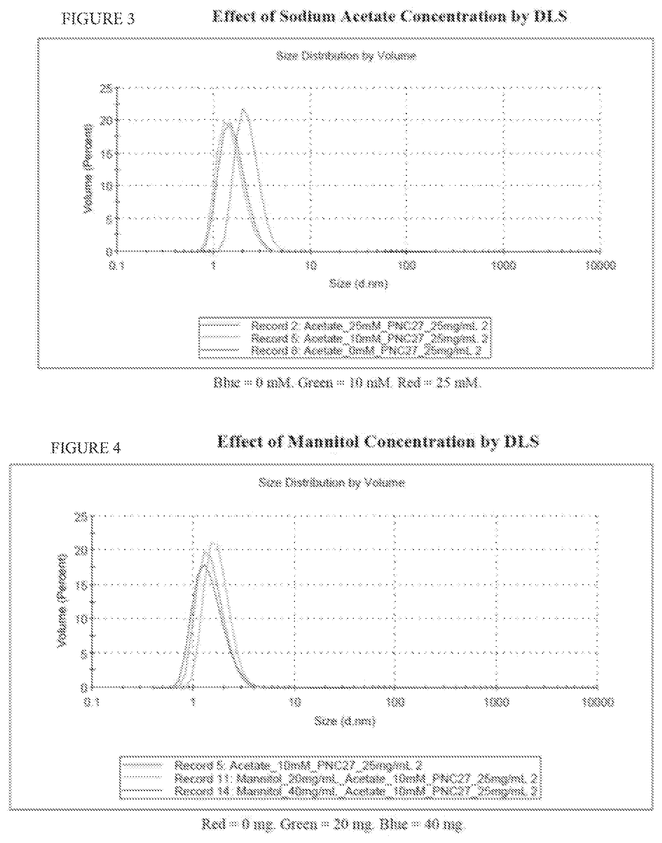

Table 2 provides a summary of the effect of buffer type and tonicity agent on physical stability. All formulations contained 25 mg/mL PNC-27 at pH 5. The formulations were stored at 2-8° C. and 40° C. and analyzed for appearance (solution vs gel), DLS (particle size), and assay content at 1 and 2 week time points. Assay data is not tabulated, however, all formulations indicated no change assay and were chemically stabile over this period. Acetate buffer appeared more stable than citrate as evidenced by citrate buffer gelling at 40° C. after 1 week and acetate stable over 2 weeks (#1 vs. #6). Increasing sodium chloride in the citrate buffer system from 50, 100, and 150 mM led to a slight increase in particle size by DLS at initial time point (#3, #4, #5 and FIG. 4). This study resulted in the selection of the composition shown in Formulation #1 using sodium acetate as buffer and mannitol as tonicity adjuster.

TABLE 5
Effect of Buffer & Tonicity on Stability of PNC-27 (25 mg/mL, pH 5)

| | | | | | | | Physical Stability | | | | | |
|---|---|---|---|---|---|---|---|---|---|---|---|---|
| | Tonicity Type | | Buffer Type | | | | Initial | | 1 week | | 2 weeks | |
| No | Mannitol | NaCl | Citrate | Acetate | Cond. | | visual | DLS | Visual | DLS | Visual | DLS |
| 1 | 40 mg | | | 10 mM | 2-8° | C. | CCS | 0.74 | CCS | 1.4 | CCS | 1.7 |
| | | | | | 40° | C. | | | CCS | 2317^{(a)} | CCS | 2.3 |
| 2 | 40 mg | | 50 mM | | 2-8° | C. | S | NT | S | NT | S | NT |
| | | | | | 40° | C. | | | S | NT | S | NT |
| 3 | | 50 mg | 10 mM | | 2-8° | C. | CCS | 9.7 | CCS | 11.7 | S | NT |
| | | | | | 40° | C. | | | Gel | NT | NT | NT |
| 4 | | 100 mg | 10 mM | | 2-8° | C. | CCS | 10.9 | CCS | 12 | S | NT |
| | | | | | 40° | C. | | | G/S | NT | G/S | NT |
| 5 | | 150 mg | 10 mM | | 2-8° | C. | CCS | 12.9 | S | NT | S | NT |
| | | | | | 40° | C. | | | S | NT | S | NT |
| 6 | 40 mg | | 10 mM | | 2-8° | C. | CCS | 3.7 | CCS | 2.7 | CCS | 3.6 |
| | | | | | 40° | C. | | | G/S | NT | C/S | NT |

^{(a)}The high reported result for the 1 week time point may be an outlier in the measurement as the 2 week sample recovers to the initial size range similar to the 2-8° C.
CCS = clear colorless solution.
G = Gel.
S = Solids formed.
NT = not tested due to solids and/or gelling instability.

### Optimization of Acetate and Mannitol

The previous studies identified the lead formulation 25 mg/mL PNC-27 in 10 mM acetate and 40 mg mannitol at pH 5.0. To further explore the parameters of this lead formulation, seven formulations were chosen to study the effects of concentration of the components as determined by DLS at an initial time point.

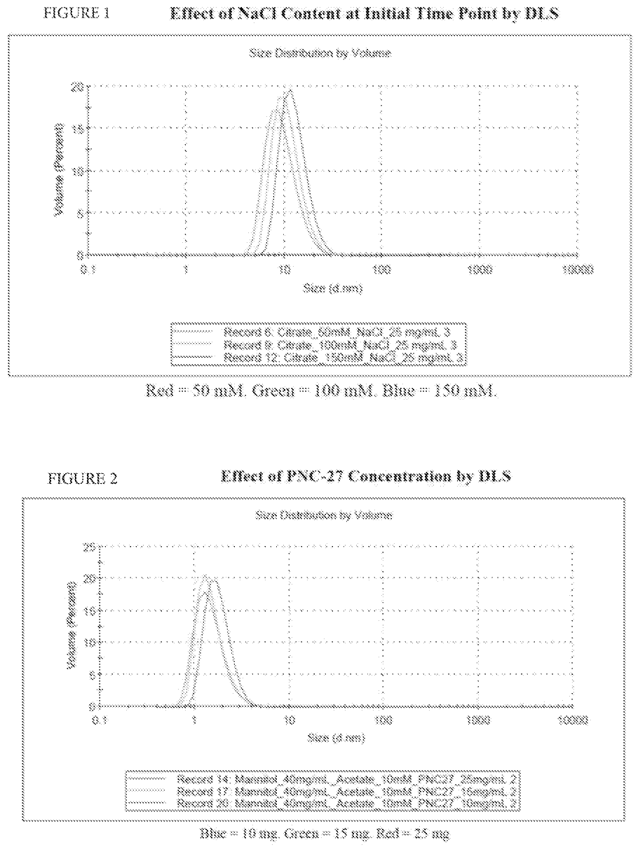

Table 3 summarizes the formulations studied and resulting DLS data. Varying the amounts of PNC-27 (#5, #6, #7 and FIG. 2), sodium acetate (#1, #2, #3 and FIG. 3), or mannitol (#2, #4, #5 and FIG. 4) vs. the lead formulation in entry 5 (same composition as Table 1) did not greatly impact aggregation state suggesting these concentration ranges to be acceptable. This information and other studies led to the progression of the lead formulation to more comprehensive development and stability studies.

TABLE 6
Effect of Concentration of PNC-27,
Acetate, Mannitol by DLS (pH 5.0)

| No | PNC-27 | Acetate | Mannitol | Particle Size (nm) |
|---|---|---|---|---|
| 1 | 25 mg | | | 1.6 |
| 2 | 25 mg | 10 mM | | 1.5 |
| 3 | 25 mg | 25 mM | | 2.3 |
| 4 | 25 mg | 10 mM | 20 mg | 1.8 |
| 5 | 25 mg | 10 mM | 40 mg | 1.5 |
| 6 | 25 mg | 10 mM | 40 mg | 1.5 |
| 7 | 25 mg | 10 mM | 40 mg | 1.8 |

TABLE 7
Proposed Specifications for PNC-27 Drug Product

| Test | Method | Limit |
|---|---|---|
| Appearance Solution * | Visual | Clear colorless solution |
| Appearance package | Visual | Clear glass vial containing a |
| | | clear colorless solution |
| Identity | HPLC | The retention time of PNC-27 |
| | | peak in sample solution |
| | | conforms with that of the |
| | | reference standard |
| Assay* (w/w peptide | HPLC | 90.0-110.0% |
| content) | | (22.5-27.5 mg/mL) |
| Related Substance | HPLC | Report Individual impurities > |
| Impurities* | | 0.2 area % |
| | | No individual impurity > 1.5 |
| | | area % |
| | | Total ≤ 5 area % |
| pH * | USP<791> | Report result |
| | | Target 4.8-5.2 |
| Osmolality * | USP <785> | Report Result |
| | | Target 270-300 mOsm/kg |
| Particulate Matter * | USP<788> | MNT 6000 particles/container ≥ |
| | | 10 μm |
| | | NMT 600 particles/container ≥ |
| | | 25 μm |
| Aggregation State * | Dynamic Light | Report results Target Range |
| | Scattering | 1.0-2.0 nm |
| | (DLS) | |
| Sterility | USP <71> | Meet the Requirements |
| Bacterial Endotoxins | USP <85> | No more than 1.0 EU/mg |

* Indicates tests performed as part of stability program

1. A stable, liquid formulation of an anti-cancer peptide, said formulation comprising 15-75 mg/ml of an anti-cancer peptide comprising an HDM-2 binding component and a membrane resident component, 10-20 mM acetate buffer, 20-60 mg/ml mannitol, and 0-30% sucrose; wherein the formulation has a pH of about 4.0-6.0.

2. The stable, liquid formulation according to claim 1, wherein said anti-cancer peptide comprises SEQ ID NO: 1.

3. The stable, liquid formulation according to claim 1, said formulation comprising about 25 mg/ml of the anti-cancer peptide.

4. The stable, liquid formulation according to claim 1, wherein said acetate buffer comprises sodium acetate trihydrate.

5. The stable, liquid formulation according to claim 1, further comprising at least one of sodium chloride and sucrose.

6. The stable, liquid formulation according to claim 1, said formulation comprising about 25 mg/ml of a the anti-cancer peptide and about 10 mM sodium acetate trihydrate, wherein the liquid formulation has a pH of about 5.1.

7. The stable, liquid formulation according to claim 1, said liquid formulation further comprising one or more of starch, glucose, dextrose, lactose, sucrose, gelatin, emulsifier, silica gel, sodium stearate, glycerol monostearate, polysorbate, sodium chloride, glycerol, propylene, glycol, and ethanol.

8. A stable, liquid formulation of an anti-cancer peptide, said formulation comprising 15-75 mg/ml of an anti-cancer peptide comprising an HDM-2 binding component and a membrane resident component, 10-20 mM acetate buffer, 20-60 mg/ml mannitol, about 20% sucrose, about 100 mM NaCl, and about 0.1% polysorbate 20; wherein the formulation has a pH of about 4.0-6.0.

9. The stable, liquid formulation according to claim 1, wherein the liquid formulation comprises no particles greater than about 2.0 nm.

10. The stable, liquid formulation according to claim 9, wherein the liquid formulation comprises no particles greater than about 3.0 nm.

11. The stable, liquid formulation according to claim 10, wherein the liquid formulation comprises no particles greater than about 5.0 nm.

12. The stable, liquid formulation according to claim 11, wherein the formulation does not form a gel upon incubation at 40° C. for two weeks.

13. The stable, liquid formulation according to claim 12, wherein less than about 1% of the anti-cancer peptide is degraded after 2 years of storage at about 2-8° C.

14. The stable, liquid formulation according to claim 13, wherein less than about 1% of the anti-cancer peptide is degraded after 3 years of storage at about 2-8° C.

15. The stable liquid formulation according to claim 14, wherein less than about 1% of the anti-cancer peptide is degraded after 5 years of storage at about 2-8° C.

16. A stable, lyophilized formulation of an anti-cancer peptide prepared by lyophilizing an aqueous formulation comprising a anti-cancer peptide, wherein the peptide comprises an HDM-2 binding component, a membrane resident component, acetate, and mannitol; wherein the formulation has a pH of about 4.0-6.0; and wherein said formulation is reconstitutable with a liquid to become a particle-free solution containing about 20-30 mg/ml concentration of the anti-cancer peptide within about 2 minutes or less.

17. The stable, lyophilized formulation according to claim 16, wherein particle-free solution comprises no particles greater than about 2.0 nm.

18. The stable, lyophilized formulation according to claim 17, wherein the particle-free solution comprises no particles greater than about 3.0 nm.

19. The stable, lyophilized formulation according to claim 18, wherein the particle-free solution comprises no particles greater than about 5.0 nm.

20. The stable, lyophilized formulation according to claim 19, wherein said formulation is stable at about 2-8° C. for at least 3 months.

21. The stable, lyophilized formulation according to any one of claim 20, wherein said formulation is stable at about 2-8° C. for at least 1 year.

22. The stable, lyophilized formulation according to claim 21, wherein less than about 1% of the anti-cancer peptide is degraded after about 2 years of storage at about 2-8° C.

23. The stable, lyophilized formulation according to claim 22, wherein less than about 1% of the anti-cancer peptide is degraded after about 3 years of storage at about 2-8° C.

24. The stable, lyophilized formulation according to claim 23, wherein less than about 1% of the anti-cancer peptide is degraded after about 5 years of storage at about 2-8° C.


SEQUENCE LISTING (56 sequences)

SEQ ID NO: 1 - protein, 15 aa, Artificial Sequence
  Synthetic Sequence
PPLSQETFSDLWKLL

SEQ ID NO: 2 - protein, 10 aa, Artificial Sequence
  Synthetic Sequence
ETFSDLWKLL

SEQ ID NO: 3 - protein, 12 aa, Artificial Sequence
  Synthetic Sequence
MPRFMDYWEGLN

SEQ ID NO: 4 - protein, 12 aa, Artificial Sequence
  Synthetic Sequence
VQNFIDYWTQQF

SEQ ID NO: 5 - protein, 12 aa, Artificial Sequence
  Synthetic Sequence
TGPAFTHYWATF

SEQ ID NO: 6 - protein, 15 aa, Artificial Sequence
  Synthetic Sequence
IDRAPTFRDHWFALV

SEQ ID NO: 7 - protein, 15 aa, Artificial Sequence
  Synthetic Sequence
PRPALVFADYWETLY

SEQ ID NO: 8 - protein, 15 aa, Artificial Sequence
  Synthetic Sequence
PAFSRFWSDLSAGAH

SEQ ID NO: 9 - protein, 10 aa, Artificial Sequence
  Synthetic Sequence
  misc_feature (2)..(2): Xaa can be any naturally occurring amino acid
  misc_feature (4)..(4): Xaa can be any naturally occurring amino acid
  misc_feature (8)..(9): Xaa can be any naturally occurring amino acid
PXFXDYWXXL

SEQ ID NO: 10 - protein, 12 aa, Artificial Sequence
  Synthetic Sequence
QPTFSDYWKLLP

SEQ ID NO: 11 - protein, 15 aa, Artificial Sequence
  Synthetic Sequence
  misc_feature (7)..(7): Xaa can be any naturally occurring amino acid
  misc_feature (14)..(14): Xaa can be any naturally occurring amino acid
PPLTSFXEYWALLXP

SEQ ID NO: 12 - protein, 15 aa, Artificial Sequence
  Synthetic Sequence
PPLSQTSFAEYWNLL

SEQ ID NO: 13 - protein, 12 aa, Artificial Sequence
  Synthetic Sequence
LTFEHYWAQLTS

SEQ ID NO: 14 - protein, 12 aa, Artificial Sequence
  Synthetic Sequence
TSFAEYWNLLSP

SEQ ID NO: 15 - protein, 12 aa, Artificial Sequence
  Synthetic Sequence
QETFSDLWKLLP

SEQ ID NO: 16 - protein, 12 aa, Artificial Sequence
  Synthetic Sequence
MPRFMDYWEGLN

SEQ ID NO: 17 - protein, 12 aa, Artificial Sequence
  Synthetic Sequence
QQMHLMSYAPGP

SEQ ID NO: 18 - protein, 12 aa, Artificial Sequence
  Synthetic Sequence
TIRPSTTMDSPT

SEQ ID NO: 19 - protein, 12 aa, Artificial Sequence
  Synthetic Sequence
YANPQMEKAFES

SEQ ID NO: 20 - protein, 12 aa, Artificial Sequence
  Synthetic Sequence
LTFEHYWAQLTS

SEQ ID NO: 21 - protein, 12 aa, Artificial Sequence
  Synthetic Sequence
LPNLTWALMPGA

SEQ ID NO: 22 - protein, 12 aa, Artificial Sequence
  Synthetic Sequence
YANPQMEKAFAS

SEQ ID NO: 23 - protein, 12 aa, Artificial Sequence
  Synthetic Sequence
LTFEHYWAQLTS

SEQ ID NO: 24 - protein, 12 aa, Artificial Sequence
  Synthetic Sequence
LLADTTHHRPWT

SEQ ID NO: 25 - protein, 17 aa, Artificial Sequence
  Synthetic Sequence
KKWKMRRNQFWVKVQRG

SEQ ID NO: 26 - protein, 13 aa, Artificial Sequence
  Synthetic Sequence
MPFSTGKRIMLGE

SEQ ID NO: 27 - protein, 14 aa, Artificial Sequence
  Synthetic Sequence
YGRKKRRQRRRPPQ

SEQ ID NO: 28 - protein, 13 aa, Artificial Sequence
  Synthetic Sequence
GRKKRRQRRRPPQ

SEQ ID NO: 29 - protein, 13 aa, Artificial Sequence
  Synthetic Sequence
GAAAAAAAAAPPQ

SEQ ID NO: 30 - protein, 7 aa, Artificial Sequence
  Synthetic Sequence
PKKKRKV

SEQ ID NO: 31 - protein, 16 aa, Artificial Sequence
  Synthetic Sequence
KRPAAIKKAGQAKKKK

SEQ ID NO: 32 - protein, 17 aa, Artificial Sequence
  Synthetic Sequence
TRQARRNRRRRWRERQR

SEQ ID NO: 33 - protein, 15 aa, Artificial Sequence
  Synthetic Sequence
RRRRNRTRRNRRRVR

SEQ ID NO: 34 - protein, 19 aa, Artificial Sequence
  Synthetic Sequence
KMTRAQRRAAARRNRWTAR

SEQ ID NO: 35 - protein, 13 aa, Artificial Sequence
  Synthetic Sequenc
TRRQRTRRARRNR

SEQ ID NO: 36 - protein, 19 aa, Artificial Sequence
  Synthetic Sequence
KLTRAQRRAAARKNKRNTR

SEQ ID NO: 37 - protein, 17 aa, Artificial Sequence
  Synthetic Sequence
NAKTRRHERRRKLAIER

SEQ ID NO: 38 - protein, 22 aa, Artificial Sequence
  Synthetic Sequence
MDAQTRRRERRAEKQAQWKAAN

SEQ ID NO: 39 - protein, 18 aa, Artificial Sequence
  Synthetic Sequence
TAKTRYKARRAELIAERR

SEQ ID NO: 40 - protein, 16 aa, Artificial Sequence
  Synthetic Sequence
TRRNKRNRIQEQLNRK

SEQ ID NO: 41 - protein, 12 aa, Artificial Sequence
  Synthetic Sequence
SQMTRQARRLYV

SEQ ID NO: 42 - protein, 26 aa, Artificial Sequence
  Synthetic Sequence
KRRIRRERNKMAAAKSRNRRRELTDT

SEQ ID NO: 43 - protein, 28 aa, Artificial Sequence
  Synthetic Sequence
RIKAERKRMRNRIAASKSRKRKLERIAR

SEQ ID NO: 44 - protein, 22 aa, Artificial Sequence
  Synthetic Sequence
KRARNTEAARRSRARKLQRMKQ

SEQ ID NO: 45 - protein, 18 aa, Artificial Sequence
  Synthetic Sequence
KLALKLALKALKAALKLA

SEQ ID NO: 46 - protein, 18 aa, Artificial Sequence
  Synthetic Sequence
LLIILRRRIRKQAKAHSK

SEQ ID NO: 47 - protein, 8 aa, Artificial Sequence
  Synthetic Sequence
RRRRRRRR

SEQ ID NO: 48 - protein, 32 aa, Artificial Sequence
  Synthetic
PPLSQETFSDLWKLLKKWKMRRNQFWVKVQRG

SEQ ID NO: 49 - protein, 27 aa, Artificial Sequence
  Synthetic Sequence
ETFSDLWKLLKKWKMRRNQFWVKVQRG

SEQ ID NO: 50 - protein, 30 aa, Artificial Sequence
  Synthetic Sequence
MPFSTGKRIMLGEKKWKMRRNQFWVKVQRG

SEQ ID NO: 51 - protein, 30 aa, Artificial Sequence
  Synthetic Sequence
TIEDSYRKQVVIDKKWKMRRNQFWVKVQRG

SEQ ID NO: 52 - protein, 13 aa, Artificial Sequence
  Synthetic Sequence
TIEDSYRKQVVID

SEQ ID NO: 53 - protein, 10 aa, Artificial Sequence
  Synthetic Sequence
GCCACCATGG

SEQ ID NO: 54 - protein, 64 aa, Artificial Sequence
  Synthetic Sequence
AGTCGAATTCGCCACCATGGAAACATTTTCAGACCTATGGAAACTACTTTGAGCGGCCGCAGTC

SEQ ID NO: 55 - protein, 10 aa, Artificial Sequence
  Synthetic Sequence
ETFSDLWKLL

SEQ ID NO: 56 - protein, 9 aa, Artificial Sequence
  Synthetic Sequence
PPLSQETFS